(19) United States
(12) Patent Application Publication
(10) Pub. No.: US 2025/0072922 A1
(43) Pub. Date: Mar. 6, 2025

(54) Surgical Clamping Device

(71) Applicants: John Meisenheimer, VII, Tampa, FL (US); John Long Meisenheimer, JR., Orlando, FL (US)

(72) Inventors: John Meisenheimer, VII, Tampa, FL (US); John Long Meisenheimer, JR., Orlando, FL (US)

(21) Appl. No.: 18/457,982

(22) Filed: Aug. 29, 2023

(51) Int. Cl. A61B 17/28

(52) U.S. Cl. CPC A61B 17/282

(57) ABSTRACT

Described are surgical clamping devices, which may include a pair of shaft members, each of which may include a jaw member extending from a proximal end of the shaft member and a finger grip extending from an opposing distal end of the shaft member. In addition, each jaw member may include a tip means for holding and clamping a patient's skin along an edge of a wound. The tip means may include at least two pointed teeth that are angled inward and downward from the shaft member. The surgical clamping device may be removed and applied for multiple stages of Mohs micrographic surgery.

### FIELD OF THE INVENTION

The field of the invention relates to a method, apparatus, and system for wound closure and hemostasis. In particular, the device may be used between stages of Mohs micrographic surgery to achieve hemostasis and apply tension to the wound edges with removal prior to the final closure of the wound.

### BACKGROUND

In many medical situations, it is often necessary to temporarily close a wound to prevent bleeding. In such situations, it may be impractical to suture or staple a wound closed due to the time involved or the need to re-open to wound.

As an example, U.S. Pat. No. 11,172,826 describes a type of surgery referred to as a Mohs micrographic surgery, named after the surgeon who invented the procedure in the 1930's. The goal of the surgery is to remove skin cancer, a type of epithelial cancer, from the face by removing all of the cancer without removing too much healthy skin from the face. To do so, a surgeon removes an area of tissue that surrounds the skin cancer, which often includes some amount of healthy tissue. During the procedure, a patient waits while a surgeon analyzes the removed tissue. The analysis often involves histological preparations of the tissue for microscopic analysis, which is often 20-30 minutes, but can take up to one hour. The tissue is examined to determine if the boundary of tissue surrounding the skin cancer also contains cancer or is free from cancer. If some cancer remains, an additional surgical procedure is performed to remove more surrounding tissue. This additional tissue is similarly analyzed for the presence of cancer cells. Each time the process is performed, it is referred to as a “stage.” The process is repeated until all cancer has been removed. The benefits of Mohs micrographic surgery include smaller defect sizes and a lower tumor recurrence rate, as compared to standard excision. Because of these benefits, Mohs surgery is often preferable for resectioning many facial and neck tumors.

Achieving adequate hemostasis between stages of Mohs surgery is challenging for many reasons. Often skin cancer patients are elderly, and there is a high degree of overlap between patients undergoing Mohs surgery and patients on anticoagulant and antiplatelet medication. Cessation of anticoagulant therapy for skin surgery is contraindicated as the serious cardiovascular event risks outweigh the extra bleeding during surgery. Due to the nature of microscopic examination between stages, typical cautery through heat or electrical destruction of tissue cannot be used, as it results in “cautery artifacts,” which obscures the surgeon's ability to examine tissue slides for residual tumor properly. In addition, patients undergoing Mohs surgery are under local anesthesia. As such, the patients are able to move and walk around between stages, which can cause shear forces between the wound defect and dressing, resulting in more bleeding.

Conventionally, hemostasis is achieved between Mohs surgery stages through pressure dressings, in which gauze is applied to the wound and held in place by an elastic bandage under tension. The success of this technique is often limited by the skill of the practitioner applying the dressing and the degree of anticoagulation. While the amount of blood loss between stages of Mohs surgery generally does not pose a risk to a patient's health, increased bleeding nevertheless results in anesthesia becoming “washed” out, which may require additional anesthesia injections. Excessive bleeding also results in time-consuming re-applications of saturated pressure dressings and undue distress for the conscious patient.

In many cases, pressure bandaging alone is inadequate for excellent hemostasis between stages of Mohs surgery. Poor hemostasis with pressure dressings leads to a waste of resources in nursing staff, in both time spent during reapplication of the bandages and time spent providing additional anesthesia. As a result, it is desirable to apply a clamping device beneath the pressure bandaging to achieve the desired amount of hemostasis.

In other fields, clamping devices have been utilized to achieve temporary wound closure. For example, U.S. Publication No. 2019/0290276 describes a wound closure device that includes skin penetrating means for anchoring the device with a pressure bar along each edge, and a locking device to maintain the device in the closed position. These types of wound closure devices work well for emergency situations, such as military operations or civilian disaster situations. But these wound closure devices are too large and/or heavy to be used on the head and/or face and/or under pressure dressings, especially for patients that are ambulatory between surgical stages. Moreover, the device would not work in the setting of Mohs surgery, as the wound defects are often small, around 1-2 cm in size.

Another example is described in U.S. Pat. No. 11,452,529, which is a surgical clamp that holds tissue steady without tearing and provides a tamponade effect. This clamping device is typically used with cervical and/or uterine procedures to hold the cervix in place. This process requires the teeth of the clamping device to have contact with each other when closed (aka, a “scissoring action”) to provide traction, along with an additional tamponade effect to reduce bleeding when the clamping device is disengaged. Because the teeth touch each other to create the “scissoring action” and pressure plates press against the clamped tissue, the clamping device damages the clamped tissue, thereby rendering the tissue inappropriate for examination as required for Mohs surgery. Moreover, the clamping device is not configured to lie flat against a patient's head or face when closed, so the clamping device would be unable to be used effectively under pressure dressing to synergize with the hemostatic effect of the pressure dressing. The clamping device is also too large and/or heavy to be used on the head and/or face, especially for patients that are ambulatory between surgical stages. Finally, the clamping device primarily is designed for traction with a secondary tamponade effect to reduce bleeding when the device is disengaged, primarily because the area is permanently closed when the device is removed so there is no need to apply the device for hemostatic control between stages.

Many other devices include multiple sets of closely grouped teeth or flat bars for joining wound edges. While such device designs may be necessary for achieving hemostasis during massive hemorrhage, it is unnecessary for the amount of hemostasis needed during Mohs. In addition, the compression or multiple perforations caused by these instruments would lead to artifacts (damage to the clamped tissue) and difficulty in assessing tissue sections for residual tumor in the event the surgeon requires another stage after using the device.

Other devices often rely on tight closure of the tissue edges, with overlap of teeth or a high degree of pressure between the longitudinal edges of the device. But this degree of pressure is unnecessary for defects encountered in Mohs surgery and may cause strangulation of the wound edges. As a result, the living tissue at the wound edges may be devitalized, thereby leading to impaired healing and unfavorable cosmetic outcomes.

As a result, there is desire for a clamping device that can achieve an appropriate amount of hemostasis between surgical stages without damaging the tissue, is light enough to be used on ambulatory patients, and is designed to lay approximately flat against a patient's skin in the area where the device is being applied. Furthermore, there is a need for a device that has a single rotating axis, as compared to other devices that slide along an axis or rotate along a longitudinal edge. The simpler design allows for smoother movement in the application and, ultimately, greater efficiency in the device's application.

### SUMMARY

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings and each claim.

According to certain embodiments of the present invention, a surgical clamping device may include a pair of shaft members, each of which may include a jaw member extending from a proximal end of the shaft member and a finger grip extending from an opposing distal end of the shaft member. In some embodiments, each jaw member may include a tip means for holding and clamping a patient's skin along an edge of a wound. The tip means may include at least two pointed teeth that are angled inward and downward from the shaft member.

In some embodiments, the surgical clamping device is applied beneath pressure bandaging to achieve a desired amount of hemostasis and/or may be removed and applied for multiple stages of Mohs micrographic surgery. The at least two pointed teeth of one of the jaw members may be spaced apart from the at least two pointed teeth of the other of the jaw members, and the distance between each of the at least two pointed teeth of one of the jaw members and each of the adjacent at least two pointed teeth of the other of the jaw members may be 1-2 cm. In some cases, the surgical clamping device is shaped to substantially contour to a patient's face and/or to a patient's head.

### BRIEF DESCRIPTION OF THE DRAWINGS

FIG. 1 is a bottom view of a surgical clamping device in an open position, according to certain embodiments of the present invention.

FIG. 2 is a bottom view of the surgical clamping device of FIG. 1 in a closed position.

FIG. 3 is a side view of the surgical clamping device of FIG. 1 in a closed position.

FIG. 4 is a top view of the surgical clamping device of FIG. 1 in a closed position.

FIG. 5 is a view from a distal end of the surgical clamping device of FIG. 1 in a closed position.

FIG. 6 is a bottom view of one of the shaft members of the surgical clamping device of FIG. 1.

FIG. 7 is a side view of the shaft member of FIG. 6.

FIG. 8 is a bottom view of another of the shaft members of the surgical clamping device of FIG. 1.

FIG. 9 is a side view of the shaft member of FIG. 8.

FIG. 10 is a perspective view of the surgical clamping device of FIG. 1 attached to a patient's scalp.

### DETAILED DESCRIPTION

The subject matter of embodiments of the present invention is described here with specificity to meet statutory requirements, but this description is not necessarily intended to limit the scope of the claims. It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below.

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. This description should not be interpreted as implying any particular order or arrangement among or between various steps or elements except when the order of individual steps or arrangement of elements is explicitly described. The claimed subject matter may be used in conjunction with other existing or future technologies.

Other technical advantages may become readily apparent to one of ordinary skill in the relevant art after review of the following figures and description. Moreover, those of ordinary skill in the relevant art will recognize and appreciate that many changes can be made to the various examples of the invention described herein, while still obtaining the beneficial results of the invention.

As used throughout this document, “each” refers to each member of a set or each member of a subset of a set. Moreover, the singular forms “a,” “an” and “the” include plural references unless the context clearly dictates otherwise. Thus, for example, reference to “a fastener” can include two or more such fasteners unless the context indicates otherwise. Ranges can be expressed herein as from “about” one particular value and/or to “about” another particular value. When such a range is expressed, another example includes from the one particular value and/or to the other particular value. Similarly, when values are expressed as approximations, by use of the antecedent “about,” the particular value forms another example. Moreover, the endpoints of each of the ranges are significant both in relation to the other endpoint, and independently of the other endpoint.

As used herein, the terms “optional” or “optionally” mean that the subsequently described feature may or may not be present, and that the description includes instances where said feature is present and instances where it is not. The word “or” as used herein means any one member of a particular list and also includes any combination of members of that list.

Further, conditional language, such as, among others, “may,” “can,” “could,” or “might,” unless specifically stated otherwise or otherwise understood within the context as used, is generally intended to convey that certain examples include, while other examples do not include, certain features, elements and/or steps. Thus, such conditional language is not generally intended to imply that features, elements and/or steps are in any way required for one or more particular examples.

Directional references such as “up,” “down,” “top,” “bottom,” “left,” “right,” “front,” “back,” “rear,” and “corners,” among others, are intended to refer to the orientation as illustrated and described in the figure (or figures) to which the components and directions are referencing. Unless otherwise specifically noted, articles depicted in the drawings are not necessarily drawn to scale.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

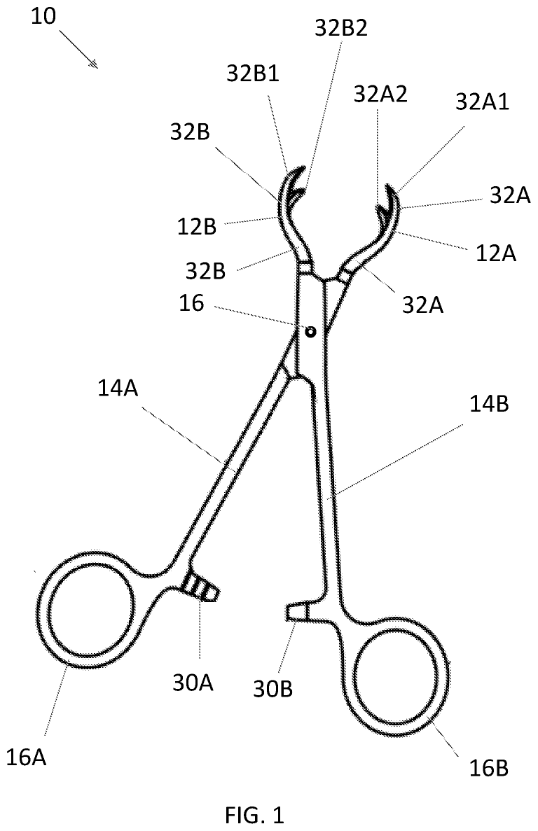

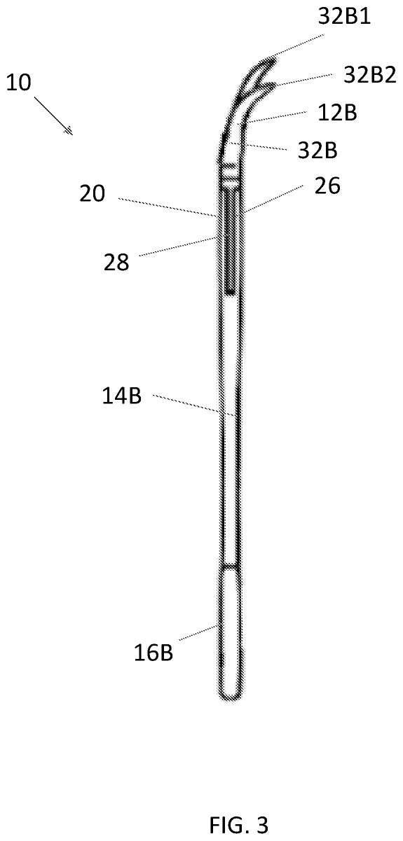

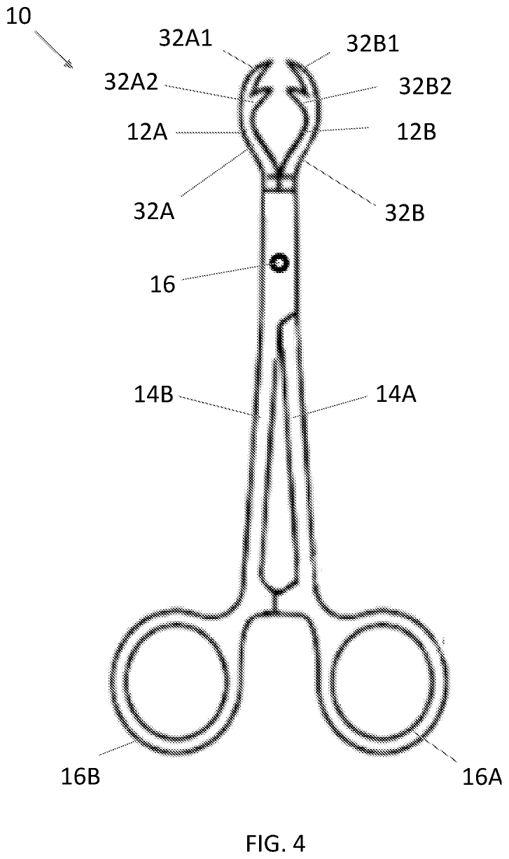

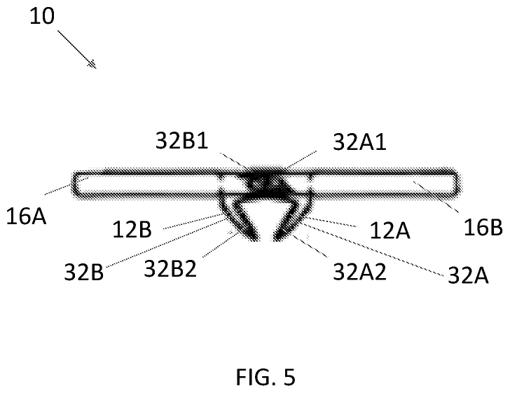

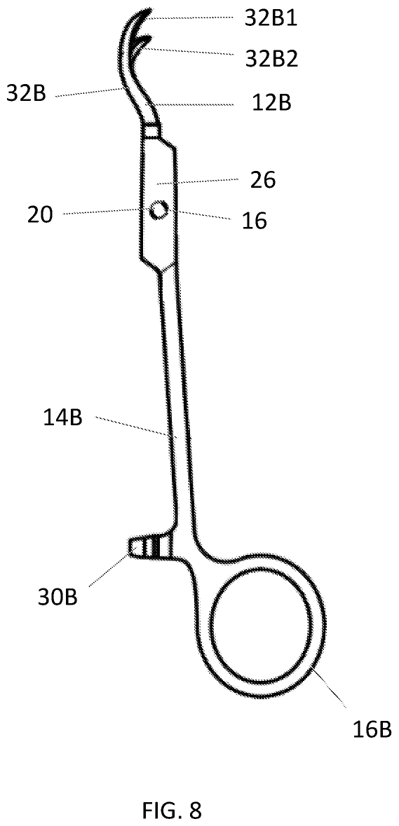

FIGS. 1-10 illustrate certain embodiments of a surgical clamping device 10. In some of these embodiments, the surgical clamping device 10 comprises opposing jaw members 12A, 12B, each of which extends from a proximal end of one of shaft members 14A, 14B. In certain embodiments, surgical clamping device 10 comprises opposing finger grips 16A, 16B, each of which extends from a distal end of one of the shaft members 14A, 14B.

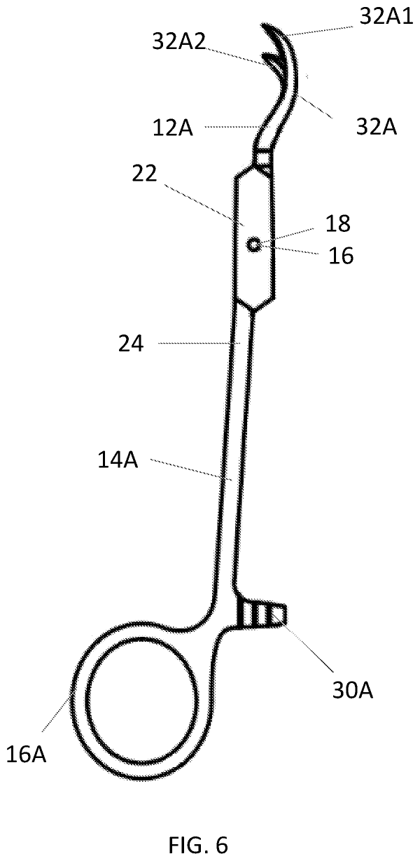

The shaft members 14A, 14B are pivotally connected to one another at pivot location 16. In certain embodiments, as best illustrated in FIGS. 6-9, the pivot location 16 comprises a projection 18 extending from one of the shaft members 14A, 14B, and a corresponding receptacle 20 located within an opposing one of the shaft members 14A, 14B.

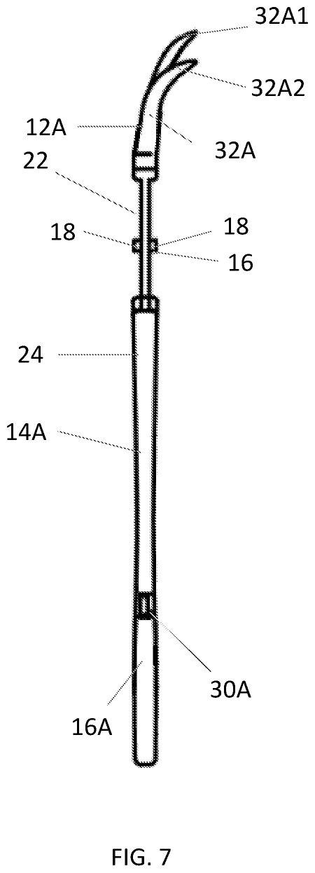

In some embodiments, as best illustrated in FIG. 7, the shaft member 14A comprises a region 22 that is reduced in thickness along at least a top side or a bottom side of the shaft member 14A proximate to the pivot location 16. In the reduced thickness region 22, the projection 18 may extend a length that is approximately equal to a difference in thickness of region 22 and a non-reduced thickness region 24. In some embodiments, as best illustrated in FIG. 7, the projection 18 extends from both sides of the region 22.

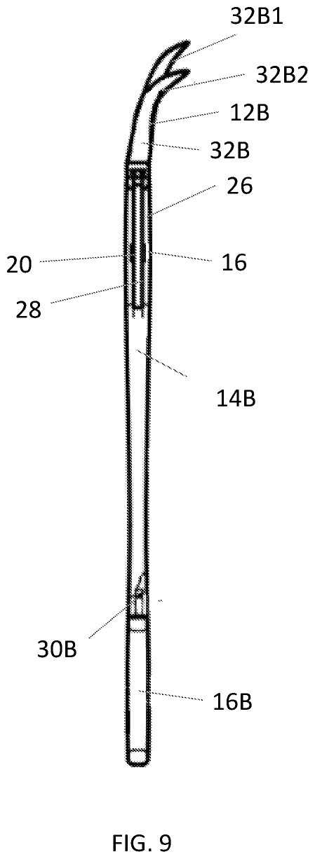

In some embodiments, as best illustrated in FIG. 9, the shaft member 14B comprises a region 26 that contains an opening 28 proximate to the pivot location 16. The opening 28 may be formed within the shaft member 14B and shaped to allow region 22 of shaft member 14A to extend therethrough when the shaft members 14A, 14B are pivotally coupled. Moreover, opening 28 may comprise the receptacle 20, which may extend at least partially through top and bottom sides of the opening 28. When the shaft members 14A, 14B are pivotally coupled, projection 18 is inserted within the receptacle 20 to prevent the shaft members 14A, 14B from disengaging.

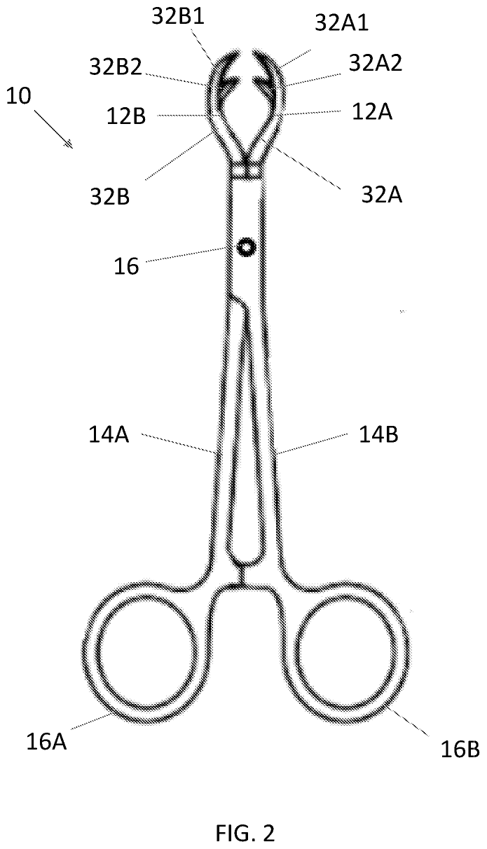

When pivotally coupled, the shaft members 14A, 14B rotate about the pivot location 16 between an open position (as best illustrated in FIG. 1) and a closed position (as best illustrated in FIGS. 2-5).

As best illustrated in FIGS. 1, 2, and 4, the surgical clamping device 10 further comprises ratchet members 30A, 30B, which are coupled to the shaft members 14A, 14B proximate the location where the finger grips 16A, 16B are coupled to each shaft member 14A. 14B. When the finger grips 16A. 16B are moved toward each other in a closing motion, ratchet member 30A, 30B interlock (as best illustrated in FIGS. 1, 2, and 4) to hold the surgical clamping device 10 in the closed position. In the closed position, a surgeon can open the surgical clamping device 10 by disengaging the ratchet members 30A, 30B.

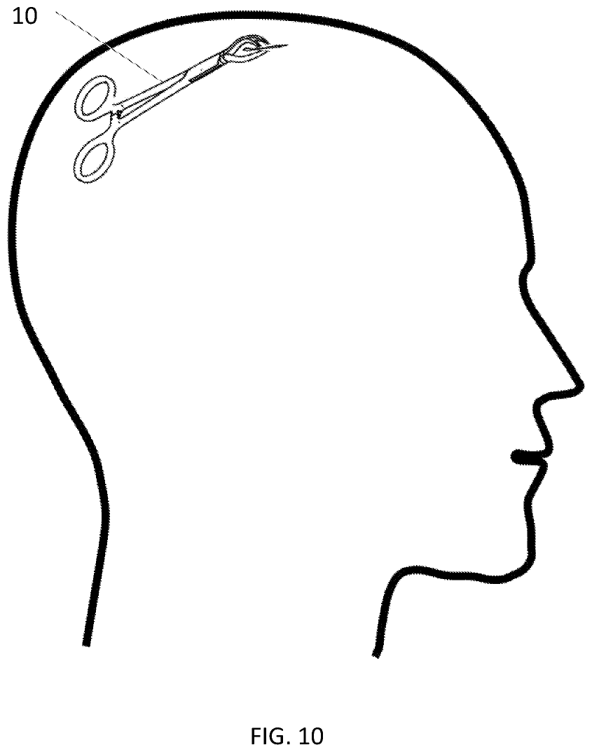

In certain embodiments, as best illustrated in FIG. 10, each jaw member 12A, 12B is provided with tip means 32A. 32B for holding and clamping a patient's skin along an edge of a wound and may be used with or without additional pressure bandaging, depending on the degree of hemostasis required.

In some embodiments, each tip means 32A, 32B is a pointed tooth 32A1, 32A2, 32B1, 32B2 that is angled inward and downward from the shaft member 14A, 14B. The teeth 32A1, 32A2, 32B1, 32B2 are positioned along edges of the wound with the surgical clamping device 10 in the open position, so that the pointed teeth 32A1, 32A2, 32B1, 32B2 superficially penetrate a patient's skin, and then the surgical clamping device 10 is closed and locked using the ratchet members 30A, 30B. As described above, tooth 32A1 may be spaced apart from 32B1, and tooth 32A2 may be spaced apart from 32B2 so that the teeth do not rub against each other when the surgical clamping device 10 is being closed. The distance between the teeth may be 1-2 cm, or any suitable distance that approximates the size of the defect being removed. The surgical clamping device 10 is designed to be used with or without pressure bandaging, depending on the degree of hemostasis required and can be easily disengaged and removed for wound closure and/or for taking further tissue stages. The surgical clamping device can be reapplied for cases that require multiple stages.

In some embodiments, the surgical clamping device 10 may have fewer or more sets or teeth than as shown in FIGS. 1-9. In other embodiments, the teeth may be set at other angles. The teeth may be set so that the space between each tooth is different than what is shown in these embodiments. Additionally, the teeth may be set so there is a greater or lesser amount of space between opposing teeth with the device in a locked position. Other embodiments may use alternative locking mechanisms, such as a clasp. Other embodiments may use a different joining mechanism, for example, two holes in each shaft with a capped pin that runs through both shafts. Other embodiments of the surgical clamping device 10 may be made from materials that cannot withstand autoclave temperature and pressure and may be disposed of after use.

The surgical clamping device 10 improves hemostasis compared to a typical pressure dressing. The surgical clamping device 10 can be applied under a usual pressure dressing due to its low-profile design, improving the effectiveness of the pressure dressing, diminishing its size, decreasing the time needed to apply the bandage, and reducing the skill level required in applying the dressing to achieve adequate hemostasis. Using the surgical clamping device 10 causes minimal additional tissue damage and has other benefits during the closure of the wound after all tumor has been removed. The design of the surgical clamping device 10 allows for static pressure distribution when bringing tissue edges together, which is imperative for use in populations with frail and elderly skin. Additionally, the time spent under tension stretches the skin, which allows for easier final closure by the surgeon.

In the following, further examples are described to facilitate the understanding of the invention:
Example A. A surgical clamping device comprising:
   a pair of shaft members, each of which comprises a jaw member extending from a proximal end of the shaft member and a finger grip extending from an opposing distal end of the shaft member;
   wherein each jaw member comprises a tip means for holding and clamping a patient's skin along an edge of a wound; and
   wherein the tip means comprises at least two pointed teeth that are angled inward and downward from the shaft member.
Example B. The surgical clamping device of any of the preceding or subsequent examples, wherein the surgical clamping device is applied beneath pressure bandaging to achieve a desired amount of hemostasis.
Example C. The surgical clamping device of any of the preceding or subsequent examples, wherein, during a multiple-stage Mohs micrographic surgery, the surgical clamping device is applied and removed following at least one stage of a multiple-stage Mohs micrographic surgery.
Example D. The surgical clamping device of any of the preceding or subsequent examples, wherein the at least two pointed teeth of one of the jaw members are spaced apart from the at least two pointed teeth of the other of the jaw members.
Example E. The surgical clamping device of any of the preceding or subsequent examples, wherein a distance between at least one of the at least two pointed teeth of one of the jaw members and at least one of the adjacent at least two pointed teeth of the other of the jaw members is 1-2 cm.
Example F. The surgical clamping device of any of the preceding or subsequent examples, wherein the surgical clamping device is shaped to substantially contour to a patient's face.
Example G. The surgical clamping device of any of the preceding or subsequent examples, wherein the surgical clamping device is shaped to substantially contour to a patient's head.
Example H. A surgical clamping device comprising:
   a pair of shaft members, each of which comprises a jaw member extending from a proximal end of the shaft member and a finger grip extending from an opposing distal end of the shaft member;
   wherein each jaw member comprises a tip means for holding and clamping a patient's skin along an edge of a wound; and
   wherein, during a multiple-stage Mohs micrographic surgery, the surgical clamping device is applied and removed following at least one stage of a multiple-stage Mohs micrographic surgery.
Example I. The surgical clamping device of any of the preceding or subsequent examples, wherein the surgical clamping device is applied beneath pressure bandaging to achieve a desired amount of hemostasis.
Example J. The surgical clamping device of any of the preceding or subsequent examples, wherein the tip means comprises at least one pointed tooth that is angled inward and downward from the shaft member.
Example K. The surgical clamping device of any of the preceding or subsequent examples, wherein the at least one pointed tooth of one of the jaw members is spaced apart from the at least one pointed tooth of the other of the jaw members.
Example L. The surgical clamping device of any of the preceding or subsequent examples, wherein a distance between the at least one pointed tooth of one of the jaw members and the at least one pointed tooth of the other of the jaw members is 1-2 cm.
Example M. The surgical clamping device of any of the preceding or subsequent examples, wherein the tip means comprises at least two pointed teeth that are angled inward and downward from the shaft member.
Example N. The surgical clamping device of any of the preceding or subsequent examples, wherein the at least two pointed teeth of one of the jaw members is spaced apart from the at least two pointed teeth of the other of the jaw members.
Example O. The surgical clamping device of any of the preceding or subsequent examples, wherein a distance between at least one of the at least two pointed teeth of one of the jaw members and at least one of the adjacent at least two pointed teeth of the other of the jaw members is 1-2 cm.
Example P. The surgical clamping device of any of the preceding or subsequent examples, wherein the surgical clamping device is shaped to substantially contour to a patient's face.
Example Q. The surgical clamping device of any of the preceding or subsequent examples, wherein the surgical clamping device is shaped to substantially contour to a patient's head.
Example R. A surgical clamping device comprising:
   a pair of shaft members, each of which comprises a jaw member extending from a proximal end of the shaft member and a finger grip extending from an opposing distal end of the shaft member;
   wherein each jaw member comprises a tip means for holding and clamping a patient's skin along an edge of a wound;
   wherein the tip means comprises at least two pointed teeth that are angled inward and downward from the shaft member;
   wherein the surgical clamping device is applied beneath pressure bandaging to achieve a desired amount of hemostasis; and
   wherein, during a multiple-stage Mohs micrographic surgery, the surgical clamping device is applied and removed following at least one stage of a multiple-stage Mohs micrographic surgery.
Example S. The surgical clamping device of any of the preceding or subsequent examples, wherein the at least two pointed teeth of one of the jaw members is spaced apart from the at least two pointed teeth of the other of the jaw members.
Example T. The surgical clamping device of any of the preceding or subsequent examples, wherein a distance between at least one of the at least two pointed teeth of one of the jaw members and at least one of the adjacent at least two pointed teeth of the other of the jaw members is 1-2 cm.

Different arrangements of the components depicted in the drawings or described above, as well as components and steps not shown or described are possible. Similarly, some features and sub-combinations are useful and may be employed without reference to other features and sub-combinations. Embodiments of the invention have been described for illustrative and not restrictive purposes, and alternative embodiments will become apparent to readers of this patent. Accordingly, the present invention is not limited to the embodiments described above or depicted in the drawings, and various embodiments and modifications may be made without departing from the scope of the claims below.

That which is claimed is:

1. A surgical clamping device comprising:
a pair of shaft members, each of which comprises a jaw member extending from a proximal end of the shaft member and a finger grip extending from an opposing distal end of the shaft member;
wherein each jaw member comprises a tip means for holding and clamping a patient's skin along an edge of a wound; and
wherein the tip means comprises at least two pointed teeth that are angled inward and downward from the shaft member.

2. The surgical clamping device of claim 1, wherein the surgical clamping device is applied beneath pressure bandaging to achieve a desired amount of hemostasis.

3. The surgical clamping device of claim 1, wherein, during a multiple-stage Mohs micrographic surgery, the surgical clamping device is applied and removed following at least one stage of a multiple-stage Mohs micrographic surgery.

4. The surgical clamping device of claim 1, wherein the at least two pointed teeth of one of the jaw members are spaced apart from the at least two pointed teeth of the other of the jaw members.

5. The surgical clamping device of claim 4, wherein a distance between at least one of the at least two pointed teeth of one of the jaw members and at least one of the adjacent at least two pointed teeth of the other of the jaw members is 1-2 cm.

6. The surgical clamping device of claim 1, wherein the surgical clamping device is shaped to substantially contour to a patient's face.

7. The surgical clamping device of claim 1, wherein the surgical clamping device is shaped to substantially contour to a patient's head.

8. A surgical clamping device comprising:
a pair of shaft members, each of which comprises a jaw member extending from a proximal end of the shaft member and a finger grip extending from an opposing distal end of the shaft member;
wherein each jaw member comprises a tip means for holding and clamping a patient's skin along an edge of a wound; and
wherein, during a multiple-stage Mohs micrographic surgery, the surgical clamping device is applied and removed following at least one stage of a multiple-stage Mohs micrographic surgery.

9. The surgical clamping device of claim 8, wherein the surgical clamping device is applied beneath pressure bandaging to achieve a desired amount of hemostasis.

10. The surgical clamping device of claim 8, wherein the tip means comprises at least one pointed tooth that is angled inward and downward from the shaft member.

11. The surgical clamping device of claim 10, wherein the at least one pointed tooth of one of the jaw members is spaced apart from the at least one pointed tooth of the other of the jaw members.

12. The surgical clamping device of claim 11, wherein a distance between the at least one pointed tooth of one of the jaw members and the at least one pointed tooth of the other of the jaw members is 1-2 cm.

13. The surgical clamping device of claim 8, wherein the tip means comprises at least two pointed teeth that are angled inward and downward from the shaft member.

14. The surgical clamping device of claim 13, wherein the at least two pointed teeth of one of the jaw members is spaced apart from the at least two pointed teeth of the other of the jaw members.

15. The surgical clamping device of claim 14, wherein a distance between at least one of the at least two pointed teeth of one of the jaw members and at least one of the adjacent at least two pointed teeth of the other of the jaw members is 1-2 cm.

16. The surgical clamping device of claim 8, wherein the surgical clamping device is shaped to substantially contour to a patient's face.

17. The surgical clamping device of claim 8, wherein the surgical clamping device is shaped to substantially contour to a patient's head.

18. A surgical clamping device comprising:
a pair of shaft members, each of which comprises a jaw member extending from a proximal end of the shaft member and a finger grip extending from an opposing distal end of the shaft member;
wherein each jaw member comprises a tip means for holding and clamping a patient's skin along an edge of a wound;
wherein the tip means comprises at least two pointed teeth that are angled inward and downward from the shaft member;
wherein the surgical clamping device is applied beneath pressure bandaging to achieve a desired amount of hemostasis; and
wherein, during a multiple-stage Mohs micrographic surgery, the surgical clamping device is applied and removed following at least one stage of a multiple-stage Mohs micrographic surgery.

19. The surgical clamping device of claim 18, wherein the at least two pointed teeth of one of the jaw members is spaced apart from the at least two pointed teeth of the other of the jaw members.

20. The surgical clamping device of claim 19, wherein a distance between at least one of the at least two pointed teeth of one of the jaw members and at least one of the adjacent at least two pointed teeth of the other of the jaw members is 1-2 cm.
